(19) United States
(12) Patent Application Publication
(10) Pub. No.: US 2013/0261409 A1
(43) Pub. Date: Oct. 3, 2013

(54) Sensing Patch Applications

(71) Applicants: Srikant Pathak, Diamond Bar, CA (US); David N. Edwards, Pasadena, CA (US)

(72) Inventors: Srikant Pathak, Diamond Bar, CA (US); David N. Edwards, Pasadena, CA (US)

(21) Appl. No.: 13/990,452

(22) PCT Filed: Nov. 30, 2010
(86) PCT No.: PCT/US10/58304

§ 371 Date: May 30, 2013

(51) Int. Cl. A61B 5/00; A61B 5/15; A61B 5/0205; A61B 5/0402; A61B 5/157; A61B 5/11; A61B 5/1477; A61B 5/145; A61B 5/01; A61B 10/00; A61B 5/1455

(52) U.S. Cl. CPC A61B 5/445; A61B 10/0045; A61B 5/150358; A61B 5/0205; A61B 5/0402; A61B 5/14551; A61B 5/1118; A61B 5/4806; A61B 5/6832; A61B 5/4839; A61B 5/742; A61B 5/0022; A61B 5/4833; A61B 5/1477; A61B 5/14546; A61B 5/14539; A61B 5/01; A61B 5/157
USPC 600/301; 600/300; 600/573; 600/362; 600/324

(57) ABSTRACT

Various methods, devices and systems for patch based physical, physiological, chemical, and biochemical sensors that diagnose and monitor disease states are described. The patch based sensors provide a panel of specific analyte parameters that determine one or more physiological conditions and/or the level of healing progression of a wound. The use of such analyte panels in local or remote monitoring of parameters related to various disease states is also described.

### FIELD OF THE INVENTION

The present invention relates to medical sensing devices, and more particularly wound care sensing devices that determine the level of parameters affecting wound healing, and indicate the status of wound healing in a patient.

### BACKGROUND OF THE INVENTION

Wound care often is labor intensive, requiring frequent attention by skilled professionals. Aging populations will increase the need for wound care. The cost of wound healing is a major concern of healthcare providers worldwide. Current approaches to treatment of wounds include improved dressings, often designed to control humidity, to keep out bacteria, and to apply antimicrobial agents and growth factors. The progress of wound healing is typically monitored by techniques such as measuring the wound diameter, color, wound depth, qualitative visual assessment and more intrusive probing to determine additional co-morbidities that may prevent the wound from healing.

For community care givers it is difficult to visually diagnose for example, a wound which is moving from colonized to critically colonized and decide when to seek specialist intervention. Generally, if the tissue bacterial concentration is greater than 10⁵ cfu (colony forming units), the tissue could be considered as being infected. Today, bacterial infection detection (through cell culture) is generally performed after a 2 week trial of topical antibiotics. Additionally, over-usage of antimicrobial products has been leading to higher costs of treatments and development of bacterial resistance (e.g. MRSA, superbug etc). Long duration of chronic wound care treatments make it necessary for a majority of care to be provided in nursing homes, long term care facility, home settings etc. Unfortunately, complications frequently develop due to inadequate treatment of wound infections in such settings.

In chronic wound care, healing is often dependent upon careful debridement, wound bed preparation and use of advanced wound care products such as dressings containing alginates, antimicrobial agents, biological agents and the like. Each of these advanced wound care products while expensive to use, is effective only under certain wound conditions and hence must be used carefully. Alginates for example, are useful for moisture management, while silver containing dressings offer no value if the wound is not infected. Collagen containing wound care products are useful only when a high protease concentration needs to be managed.

The problem of selecting and using a suitable dressing and treatment course is compounded in home care or long term care settings where there is typically less expertise and less pathology laboratory support. Therefore, there exists is a long unmet need for devices that evaluate the healing status of a wound and recommend a proper dressing and/or a course of treatment action at such care facilities.

In the US alone there are 5.5 million chronic wounds per year, from which almost 60% are treated in home care settings. Due to ageing and an increased diabetic population, the growth in chronic wounds is almost 10% per year for the foreseeable future. Typical care providers are segmented into home care, non-specialized clinics, hospital and specialized wound clinics. Swab or biopsy are the current gold standards for the microbiological analysis of wound exudates but typically require a minimum of 24 to 72 hours to perform. PCR (polymerase chain reaction) based bacterial detection is faster but requires expertise and is expensive to perform. Consequently, such tools are currently not very widespread even for wound care clinics or hospitals and are infrequently used in home care settings if at all. Accordingly, a need exists for strategies for assessing wounds especially in non-specialized settings, which lead to reduced healing time and reduced complications.

Developments in biomarkers during the past decade have enabled the use of several biomarkers from research laboratories to commercial applications such as pathology labs in hospitals. Various biomarkers are known and used to reliably measure disease progression or healing of chronic conditions by specifically measuring and correlating specific concentrations of one or more analytes. Another example of commercial applications of biomarkers is in home based diagnostic kits. These kits typically provide rapid, easy, non-invasive or at least minimally invasive assessments of health conditions, which can be obtained and/or interpreted by patients or generalist care providers. Although current applications for biomarkers and particularly commercial applications have been significant, a need remains for further use and application of biomarkers, particularly in home based or point of care diagnostic applications. Recent innovations in wireless technologies have made the use of biomarkers a ubiquitous, reliable and cost-effective tool for remote monitoring of patients for a variety of health conditions and disease management.

Certain diseases or health conditions require particular attention in monitoring and continual assessment so that appropriate treatment can be performed. Examples of such diseases or conditions include, but are not limited to, chronic wounds which are typically accompanied by life threatening conditions associated with diabetes, heart conditions and the like, acute trauma, and long term use of medication and particularly in susceptible populations.

Thus, there is a need for rapid, easy, non-invasive or minimally invasive point-of-care or home based diagnostics which can be interpreted by generalist care givers and patients. There is a further need for an easy to use device which provides useful actionable data on wound healing status for the following sectors: (a) patients with chronic wounds with co-morbidity such as diabetes, heart condition etc. (b) patients with acute trauma with co-morbidities (c) long term medication use in a susceptible population and (d) populations with heart conditions.

### BRIEF SUMMARY OF THE INVENTION

The embodiments of the present invention described herein are not intended to be exhaustive or to limit the invention to the precise forms disclosed in the following detailed description. Rather, the embodiments are chosen and described so that others skilled in the art may appreciate and understand the principles and practices of the present invention.

The present invention is directed to a wound care sensing device with a functional layer which determines the level of specific parameters from wound exudates and provides an indication on the status of the wound healing process. The specific parameters include at least one chemical or biological entities such as bacteria, inflammation cytokines, proteases, growth factors, ECM receptors, pH, iconicity, NOₓ/O₂, temperature and integrins. In one embodiment of the invention, the wound sensing device also includes a sampling layer. The sampling layer operates through electrophoresis, capillary effect, or pressure driven mechanisms.

In one embodiment of the current invention, the status of a wound healing process is indicated through a color change, or a digital display. In another embodiment of the current invention, the results of one or more analyses are captured by a handheld device. The handheld device preferably sends the results to a medical professional through wireless communication.

In one embodiment of the invention, a wound care patch contains multiple wound healing indicator devices. Each wound sensing device is activated separately when needed or as desired. In a further embodiment of the invention, the wound sensing patch also includes sensors that measure the physical properties of a patient.

In one embodiment of the invention, a method of sensing the wound healing status includes the steps of applying a wound sensing patch on the wound area of a patient, and sensing the wound status using the wound sensing patch. In a further embodiment of the invention, the wound status is further sent to a medical professional, such as a medical doctor, or a well-established medical monitoring program. Once analyzed, a recommendation is then sent back to the patient or a care giver for actions.

In a further embodiment of the invention, the compliance of a patient is monitored through the use of a wound sensing device, and financial reward or penalty is linked to the degree of patient compliance.

Other features and advantages of the present invention will become apparent to those skilled in the art from the following detailed description. It is to be understood, however, that the detailed description of the various embodiments and specific examples, while indicating preferred and other embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

These, as well as other objects and advantages of this invention, will be more completely understood and appreciated by referring to the following more detailed description of the presently preferred exemplary embodiments of the invention in conjunction with the accompanying drawings, of which:

FIG. 1 is a schematic cross sectional view of a wound sensing device.

FIG. 2 shows an exemplary use of a color coded overlay to communicate the concentration and/or severity of the measured analytes.

FIG. 3 is an exemplary device visual interface using three parameters (MMP, pH and bioburden).

FIG. 4 is a collection of exemplary actionable outcomes of a wound sensing device.

FIG. 5 is an exemplary on-demand sampling system for a sensing device. Sampling could be initiated by pulling the tab. Multiple tabs could be used to create a multi-day use device.

FIG. 6 is an exemplary multi-parameter sensing device. Different parametric concentrations have either a direct or inverse dependencies on wound healing outcome.

FIG. 7 is an exemplary panel of a multi-parameter sensing device.

FIG. 8 is an exemplary single fluid channel multi-parameter sensing device.

FIG. 9 is an exemplary multiple fluid channel multi-parameter sensing device.

FIG. 10 is an exemplary illustration of a closed loop communication.

Unless otherwise indicated, the illustrations in the noted figures are not necessarily drawn to scale.

### DETAILED DESCRIPTION OF THE EMBODIMENTS

The apparatuses, systems, and methods disclosed herein are described in detail by way of examples and with reference to the figures. Unless otherwise specified, like numbers in the figures indicate references to the same, similar, or corresponding elements throughout the figures. It will be appreciated that modifications to disclosed and described examples, arrangements, configurations, components, elements, apparatuses, methods, materials, etc. can be made and may be desired for a specific application. In this disclosure, any identification of specific shapes, materials, techniques, arrangements, etc. are either related to a specific example presented or are merely a general description of such a shape, material, technique, arrangement, etc. Identifications of specific details or examples are not intended to be, and should not be, construed as mandatory or limiting unless specifically designated as such. Selected examples of apparatuses and methods are hereinafter disclosed and described in detail with reference made to the noted figures.

The present invention relates to a wound sensing device which indicates the current status of the wound healing process. The device determines the level of parameters that affect wound healing, such as proteases, pH, bacterial bioburden etc. and provides an indication as to the status of the wound healing. The indication can be shown as a change of color, shape (lines, dots etc.) or presented using other types of notifications. Such indication prompts a care provider when a wound requires a specific type of wound care product or an intervention by a medical specialist.

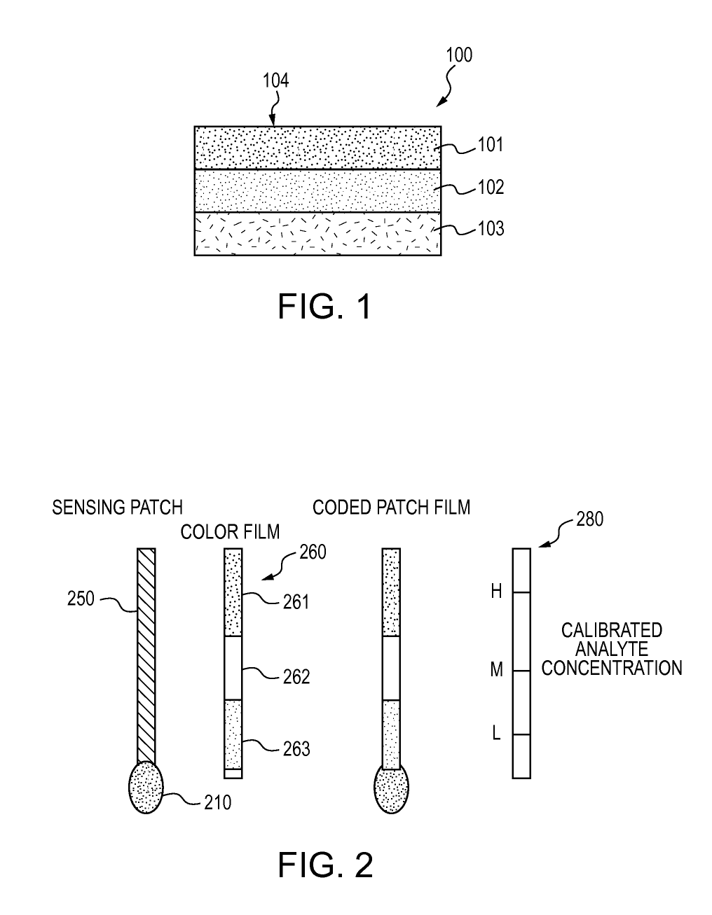

Reference is now directed to FIG. 1, which provides a sectional view of an exemplary wound sensing device 100. The wound sensing device 100 includes a carrier layer 101, a functional layer 102, and a sampling layer 103. The carrier layer 101 protects the wound sensing device and promotes ease of handling. Suitable materials for the carrier layer include but are not limited to a clear plastic film, nonwoven or woven material. Other materials suitable for a carrier layer of a medical patch can be used as the carrier layer material for the preferred embodiment devices described herein. It is preferred that the carrier layer is a transparent or semitransparent material. The carrier layer may contain instructions or visual aids on a top side 104 or which are visible through the carrier layer 101. Exemplary carrier materials include polyethylene (PE), polypropylene (PP), polyethylene terephthalate (PET), polyurethane films, preferably with a high moisture vapor transmission rate.

The functional layer 102 assesses wound healing by beneficially determining the level of analytes or parameters that are useful indicators of wound healing. The functional layer includes at least one functional component and at least one indication component. Optionally, the functional layer may include other components. The functional components react selectively with relevant analytes in the wound exudates and trigger an indication component to show the result. There are a number of mechanisms by which this functional layer operates: colorimetric, fluorescent, and electrochemical assays. In the case of colorimetric or fluorescent assays, the color change due to the reaction itself functions as the visual indication. In electrochemical assays, the functional component reacts with a specific wound analyte to give a change in current or voltage. Such electrical signals can be readily converted to visual signals or an alarm. Several enabling examples of each of the detection mechanisms are described herein.

A range of analytes or factors is typically critical to wound healing. For example, analytes or factors such as proteases such as MMP-2 and MMP-9, bacterial load, inflammation cytokines, biofilm presence, moisture content, integrins, chemokines, growth factor receptors, level of Ca²⁺/Mg²⁺, NOₓ/O₂, pH and temperature are involved or at least typically associated with wound healing. Not all of these factors need to be deficient in every patient for lack of healing in every case. The actual parameters will vary from case to case.

Several representative and enabling detection methodologies include matrix metalloproteinase (MMP), bioburden, pH and ionic measurement. These are illustrative embodiments and alternative methods of performing such assays will exist without significantly departing from the scope of the methods and approaches described herein.

The matrix metalloproteinase (MMPs) constitute a family of zinc-dependent endopeptidases that function within the extracellular matrix. These enzymes are responsible for the breakdown of connective tissues and are important in bone remodeling, repair of tissue damage and digesting extracellular matrix components. MMPs tend to have multiple substrates, with most family members having the ability to degrade several different types of collagen along with elastin, gelatin and fibronectin. Most MMPs contain three major domains—regulatory, catalytic and a hemopexin domain. The regulatory domain must be removed before the enzyme can be active. The hemopexin domain aids in enzyme binding to certain substrates, although it is not necessary for the catalytic function of the enzyme.

There are commercially available antibodies against most MMPs, such as MMP-1, 2, 3, 9 available from Molecular Probes Inc. USA. These antibodies that are directed against a stretch of amino acids forming a small hinge between the catalytic and hemopexin domains leading to high specificity and very little cross-reactivity. All of these antibodies recognize both the inactive and active forms of their respective MMP targets and are suitable for western blotting, immunoprecipitation and immunohistochemistry applications. Several modifications are available to label such antibodies with fluorescent dye, biotin or enzyme-labeled complexes of such antibodies. By properly using a secondary antibody labeled with an optical or electroactive molecule, a range of ELISA type of tests can be run leading to a detectable optical or electrochemical signature. To further enhance the signal and reduce background noise, standard signal amplification techniques could be used such as those mediated by streptavidin-biotin interactions. Additionally, a range of fluorogenic substrates could be used which fluoresce when properly activated by a binding interaction.

When using colorimetric assays, some exemplary pathways for MMPs utilize thiopeptide substrates which upon cleavage by the MMP release a sulfhydryl group, which can be detected with a color developing thiol-reactive agent, 4,4′-dithiodipyridine or Ellman's Reagent at 412 nm. Commercial products available under the designation SENSOLYTE® Generic MMP Assay Kit, and which utilize this technique are sold by Anaspec Inc. USA. These products can be used to detect the activity of a variety of MMPs, including MMP-1, 2, 3, 7, 8, 9, 12, 13, and 14 or for high throughput screening of MMPs' inducers and inhibitors.

To further improve the sensitivity of colorimetric assays, an ELISA type approach may additionally be employed. One example of ELISA based MMP assays uses HRP conjugated streptavidin. Commercially available ELISA kits for MMP-1, 3, 8, 9, 10, and 13 are available from Anaspec Inc, where the activity can be colorometrically determined at 450 nm. BIOTRAK activity assay system from GE Healthcare, USA provides another example of a commercially available colorometric sandwich type ELISA kit which utilizes chromogenic peptides and is readable at 405 nm. Yet another ELISA assay kit for Human MMP-8 is marketed by RayBiotech Inc (USA) utilizing HRP conjugated streptavidin in which the activity is measured by measuring the color at 450 nm.

It will be appreciated that almost all the examples involving ELISA type assays can be adopted in a flow through microfluidic surface for a sensing device in multiple combinations using standard molecular biology protocols for detection of MMPs.

Fluorescent assays are beneficial in instances where auto-fluorescence of the sample is expected to be low and where a better sensitivity is desired for analytes with similar binding coefficients. For direct fluorescence measurements, a dye labeled antibody is employed in direct or sandwich type ELISA assays. Typically, an antibody could be labeled by reacting with an amine and thiol reactive dyes attached to fluorophores such as ALEXA FLUOR®, FITC, fluorescein, rhodamine etc and are commercially available in numerous excitation/emission combinations from several sources such as Sigma Aldrich Inc (USA). A number of antibody/protein modification kits are also available for performing desired fluorescent labeling, such as ZENON series of products from Molecular Probes.

To further improve the sensitivity, Fluorescence Resonance Energy Transfer (FRET) based detection could be employed. The FRET substrate comprises a fluorophore and a quencher moiety separated by an amino acid sequence. Upon protease cleavage, the fluorophore separates from the quencher and is free to emit a detectable fluorescent signal. The magnitude of the resultant signal is proportional to the degree of substrate cleavage and hence could be used to quantify the concentration of MMP. ENZCHEK Peptidase/Protease Assay Kit, E33758, available from Molecular Probes, embodies this principle and once activated by MMP emits a fluorescent signal at approximately 528 nm (excitation at about 502 nm). A number of recognitive or hydrolysable amino acid tethers for various MMPs have been identified in the scientific literature and can be beneficially adapted in the sensing device for FRET based detection of MMP. Further details are set forth in G. M. McGeehan et al., Anal. Biochem, 1993, 212, 58-64.

Additional useful fluorogenic FRET substrates for detection of MMP can be based on dye labeled casein, gelatin, collagen (Type I and Type IV), and elastase—all of which are excellent substrates for various MMPs. Several such substrates are commercially available by various commercial providers or can easily be prepared by reacting the previously noted protein substrates with amine or thiol reactive dye precursors. Examples of casein based substrates for protease assay using green-fluorescent BODIPY FL (excitation at 503 nm, emission at 513 nm) and red-fluorescent BODIPY TR-X (excitation at 589 nm, emission at 617 nm) fluorescence are those available from Molecular Probes. Examples of commercially available gelatinase substrates include INNOZYME Gelatinase (MMP-2/MMP-9) Activity Assay Kit sold by EMD Chemicals, USA. All the proteins and modified proteins are commercially available from multiple sources (such as AnaSpec Inc.) and can be produced using standard molecular biology conjugation protocols.

Additionally, classic chemiluminescent systems such as luminol/peroxide can be used for detection by tagging the antibodies with appropriate enzymes.

As provided above, Fluorogenic FRET substrates can be used for detection in the sensing device with or without the need for the microfluidic separation scheme. It will be appreciated that multiple combinations of dyes, quencher and fluorogenic substrates could be beneficially used and adapted on the sensing device with or without a type flow through microfluidic system using standard molecular biology protocols for enzyme modifications and detections.

Electrochemical assays can be realized by labeling antibodies, cleavable peptides or cleavable peptide substrates with electroactive molecules such as ferrocene. The electrical activity (electrochemistry or conductivity) of the assay sample can then be modified once an MMP has acted upon the labeled substrate to cleave and release the molecule. Such an approach can also be used to quantify the MMP concentration since electrical activity is directly proportional to the free electroactive molecule in the solution. Several measurement techniques, see for example, Y. Lin et al., J. Am. Chem. Soc., 2006, 128, 12382-12383, such as cyclic voltammetry, linear sweep voltammetry and the like could be employed to obtain a measured change in voltage or current on the sensing device. Several embodiments of various conductive electrode materials known in the art could also be used.

As disclosed herein, the different modes of detection using colorimetric, fluorescent and electrical measurement employ a variety of labels, modified substrates, enzymes and approaches. Therefore, it will be appreciated that specific extension of one or more of these aspects could be used to detect cytokines, integrins and growth factors. Several reagents to enable such measurements are commercially available from GE healthcare, R&D Systems and Molecular Probes, among others. One particular example is Quantikine TNF-α/TNFSF1A Immunoassay and QuantiGlo Human TNF-α Chemiluminescent Immunoassay available from R&D Systems (USA). One or more of such systems could be adopted and used in a sensing device system and/or utilized in a detection approach.

Solution hydrogen ion concentration (pH) measurement of a wound while not always diagnostic can be a powerful tool when used in conjunction with bioburden, MMP and other measurements. An example of a relevant pH measurement is the change in pH which can serve as an indication as to the progress of healing or lack thereof. Several pH sensitive dyes or dye precursors are available and can be adapted for the sensing device applications. Several fluorescein and fluorescein derivatives, such as fluorescein sulfonic acid, carboxynapthofluorescein could be beneficially used to indicate pH changes close to neutral and are available from several commercial sources. PH sensitive fluorescent dye precursors (available from Sigma-Aldrich and others) can be used to prepare useful pH sensitive conjugates for attachment with the sensing device substrate surface. A beneficial pH range of measurement for the sensing device is preferably in the range of pH 4 to pH 9.

As is the case with pH, a number of fluorescent Ca²⁺ sensitive dyes are available that allow measurement of calcium ion concentration in an extracellular or intracellular matrix such as wound exudates. Based on modifications of classic fluorescent molecules such as Oregon Green, or substituted rhodamine or fluorescein, these commercially available materials have variable binding affinity to Ca²⁺ and can be anchored to a surface or supported in a porous matrix and provide a fluorescent signal when excited with ultraviolet or visible light. Additionally, bioluminescent Ca²⁺ indicators for example commercially available AEQUORIN (Molecular Probes) could be used. Likewise several Mg²⁺ sensitive dyes are available. It will be appreciated that it is beneficial to measure and calibrate Ca²⁺/Mg²⁺ type measurements with an appropriate pH measurement since variation in pH may affect their binding capacity and fluorescence.

A high level of bioburden may indicate a critical colonization stage of a wound leading to infection. Suitable methods for measuring the bioburden in the sensing device as previously disclosed include gram staining methods, nucleic acid stains, cell viability measurements, and ATP determination among others. Bacterial cell viability can be assessed by using a mixture of nucleic acid stains for example, SYTO 9 dye and propidium iodide to distinguish live bacteria with intact plasma membranes from dead bacteria with compromised membranes. Green fluorescent SYTO 9 stains all bacteria in a population including those with intact membranes and those with damaged membranes. In contrast, propidium iodide penetrates only bacteria with damaged membranes. Using an appropriate mixture of the SYTO 9 and propidium iodide stains, bacteria with intact cell membranes fluoresce bright green, whereas bacteria with damaged membranes exhibit significantly less green fluorescence and they often also fluoresce red. The cell type and the gram character influence the amount of red-fluorescent staining exhibited by dead bacteria. Using a 488 nm (argon-ion laser) excitation and appropriate filters, regions of bacterial populations (live or dead) can be established. Several variations of such live/dead bacterial assay are commercially available from Molecular Probes and other suppliers. SYTO 9 for gram positive, and hexidium iodide for gram negative nucleic stains can also be used for bacterial counting in such bacterial populations.

Another method of optical detection of bacterial cells is by measuring the ATP concentration released from live/proliferating bacterial cells. A luciferin-luciferase bioluminescence based assay can be incorporated within the sensing device, which produces light having a wavelength of approximately 560 nm by reaction of luciferase (enzyme) on luciferin (substrate) in the presence of ATP and oxygen. The reagents required to enable this approach are available from a number of biochemical suppliers.

Additional methodologies for detection of specific bacteria typically involve reaction with bacterial enzymes, bacterial proteins, bacteria specific polyclonal antibodies, cell surface antigens and may employ a colorimetric, fluorescent or electrochemical reporting technique. In this regard, the previous description of using a flow through system, ELISA systems, labeling approaches and detection schemes (as applied to MMP detection) can also be used in conjunction with an appropriate antibody/reporting system. In sandwich assays, secondary antibodies can be labeled with enzymes that act upon fluorogenic, chromogenic or electroactive substrates. Additionally, bacterial strain specific bacteriophages can additionally be employed as a component in sandwich assays in one or more of the detection methods.

In one embodiment the functional components are diagnostic arrays or panels that utilize detection mechanisms utilizing the specificity of bacteriophages towards known pathogenic bacteria. Bacteriophages are naturally occurring viruses that rapidly multiply by inserting genetic material to a specific bacteria (“host”) and killing the bacteria during the process. Bacteriophages are highly specific to certain strains of bacteria and can be used in sandwich assays for detecting specific strains of pathogenic bacteria.

Other mechanisms can also be used to construct the functional layer. Such mechanisms include dipstick based fluorescence assays, and lateral flow based enzyme linked immunosorbent assay (ELISA) type approaches, dielectrophoresis, free-flow electrophoresis, ATP bioluminescence, impedance, ELISA and other immunoassay methods, pH measurement, optical diffraction-based techniques, agglutination techniques, chromogenic agars, molecular imprinting for the real-time analysis, and the like.

For electrochemical detection mechanisms, the change in current or voltage can be captured by an intermediary device such as an iPHONE®, BLACKBERRY® telephone, other smart phone, or any communicable handheld device equipped with one or more appropriate sensor port(s). Conductive lines can be printed upon the sensing device to provide a physical connection to such an intermediary device. Alternatively, an active or passive RFID tag with built-in sensor port can be incorporated within the sensing device which can be programmed to read at certain thresholds and wirelessly communicate information to the display device accessible to a patient or care provider. Suppliers of usable RFID tags include Avery Dennison Inc (USA), Alien Technology, Impinj, Intermec, Motorolla, and Confidex (all of USA or significant US presence) among others. Commercially available RFID readers are available from Alien Technology, Motorola, Invengo Inc., and Symbol technologies (all of USA).

Further, a digital display can be used to provide visual information regarding the wound status. Color can be digitally generated by correlating the measured concentration of the respective analytes with a predetermined value.

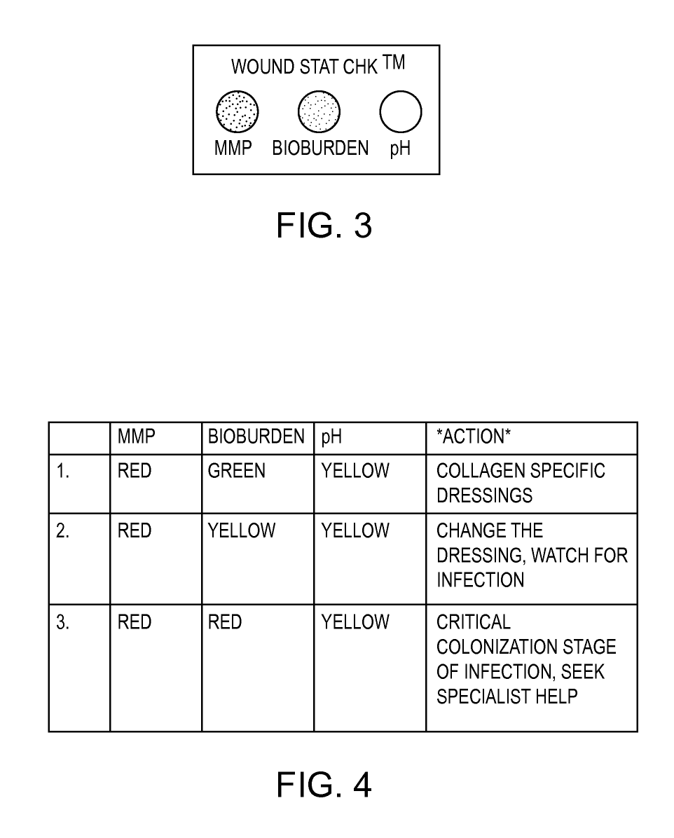

In an embodiment of coulometric assays in flow through systems, similar information can be provided by spatial overlaying of colored films on top of the measured concentration to indicate the status of wound healing. FIG. 2 is a representative schematic of such a device. A colored film strip 260 with colors 261, 262 and 263, different from each other, is positioned over a functional layer 250. Preferably, the colored film strip includes a collection of regions, each having a different color. The colored film strip also preferably defines a wound location generally denoted by 210. Upon placement of the device on a wound, the wound location is preferably located directly over the wound. Thus, by monitoring the transport or change in position of wound exudates relative to the location of the wound location and various regions having different colors along the film strip, indication is readily provided as to movement, rate of movement, and other aspects of the exudates and the wound. As the wound exudates 210 move along the functional layer, the band of color that is reached by the exudates provides visual indication of the wound healing status. An optional standard indicator spot or lane 280 may be provided to ensure the reliability of such devices in a given measurement. FIG. 3 shows an exemplary outcome of such analysis. MMP, bioburden and pH are measured and indicated with one or more colors. FIG. 4 shows an exemplary table of actionable outcomes. Such devices can also provide feedback by using electrochemical sandwich titrations where the measurement can be electronically manipulated and displayed on a handheld device.

Many biosensors include a sensing layer associated with a transducer. The sensing layer interacts with a medium including one or more targeted analytes. The sensing layer can include a material that can bind to the analytes such as an enzyme, an antibody, a chemical or biological receptor, a microorganism, a nucleic acid, and the like. Upon binding of the analytes with the sensing layer, a physicochemical signal induces a change in the transducer. The change in the transducer permits a measurement that can be optical (e.g., a viewable diffraction pattern or change in color), potentiometric, gravimetric, amperometric, conductimetric, calorimetric, acoustic, and the like. The preferred embodiment sensing devices described herein can employ one or more of such transducers. Additional description is provided in “Modern Topics in Chemical Sensing,” Chemical Reviews, 2008, 108(2).

To accomplish the various modes of electrochemical detection in the sensing device-many forms of electrodes can be incorporated within the embodiments of the present invention. The electrodes can be created with photolithography, printing technologies such as inkjet or screen printing, mechanical assembly, any technique suitable in the production of semiconductor chips, and the like. Further, inks can include inorganic agents (carbon black, silver etc.) or organic agents (fluorescent dyes, linkers etc.) or biochemical agents (protein fragments, nucleic acids, haptens etc.). Furthermore, the method of printing may beneficially include printing and patterning of one or more layers of such inks as described in “Solution Processing of Inorganic Material” David B. Mitzi Ed., Wiley Publication, 2009, pp 379-406.

In one preferred embodiment, the preferred device provides visual feedback by coulometric or luminescence assays. Luminescence assays can include direct fluorescence detection, fluorescence resonance energy transfer or bioluminescent approaches. In yet another embodiment the coulometric or luminescence titrations are applied to liquid, gases or odors (all of which are “fluids”) emanating from the non-healing wound as a result of infection.

Additional strategies of visually indicating an individual analyte's concentration include but are not limited to the use of bars, spots, signs, line and the likes to provide the end-user information concerning the status of the wound. In yet another embodiment the device is used by a trained medical staff with or without the attached outcome table.

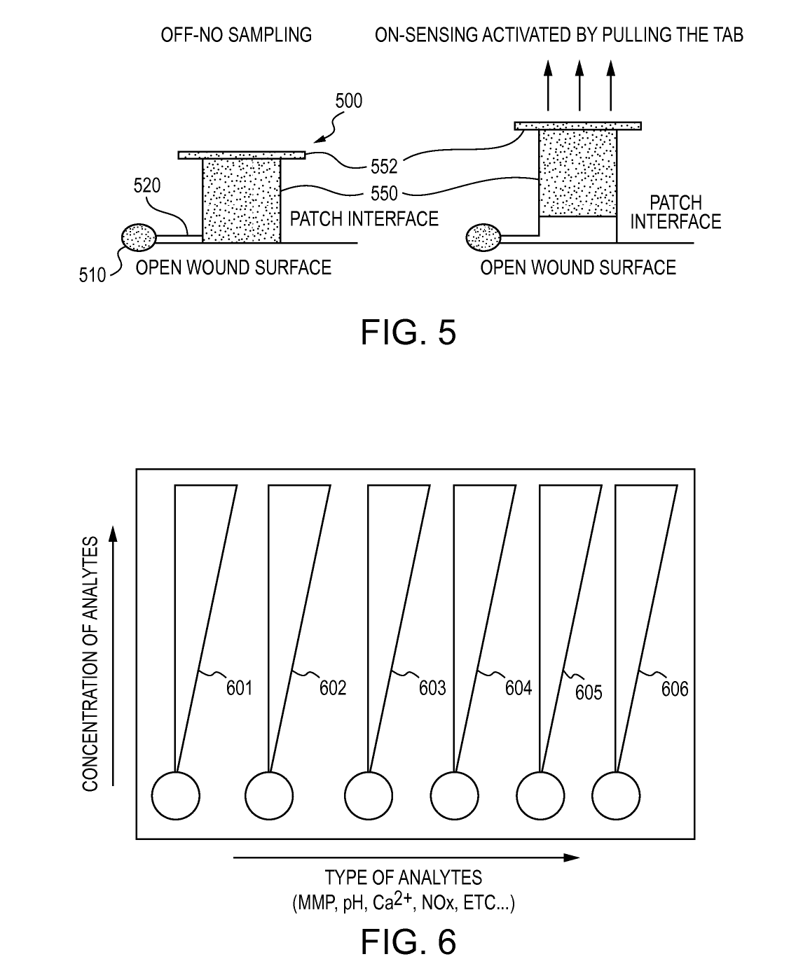

Referring again to FIG. 1, the sampling layer 103 is preferably placed in contact with the wound site of a patient. The sampling layer draws wound exudates from the wound site, and passes the exudates on to the functional layer 102. The sampling mechanism can be through a pump action or through vacuum creation. For example, vacuum suction can be created through the use of microfluidic sampling channels. The microfluidic sampling channels can be first sealed or gated with a tab or a membrane at one end of the channels. FIG. 5 is a schematic illustration of such a system 500. A microfluidics sample channel 550 includes a tab 552 at one end of the channel. The other end which forms a sampling port 520 is in contact with wound exudates 510. The sampling can be activated by pulling the tab 552 to create a pressure gradient in the channel 550. Such pressure gradient will begin sampling of the fluid through the port 520. Creation of such pressure gradient can also be accomplished through the action of physically rupturing a wall of the microfluidic sample channel for example by puncturing a membrane, or electrical means such as by applying an electrical field over the microfluidic sample channel to induce particle movement, for example such as electrophoresis etc.

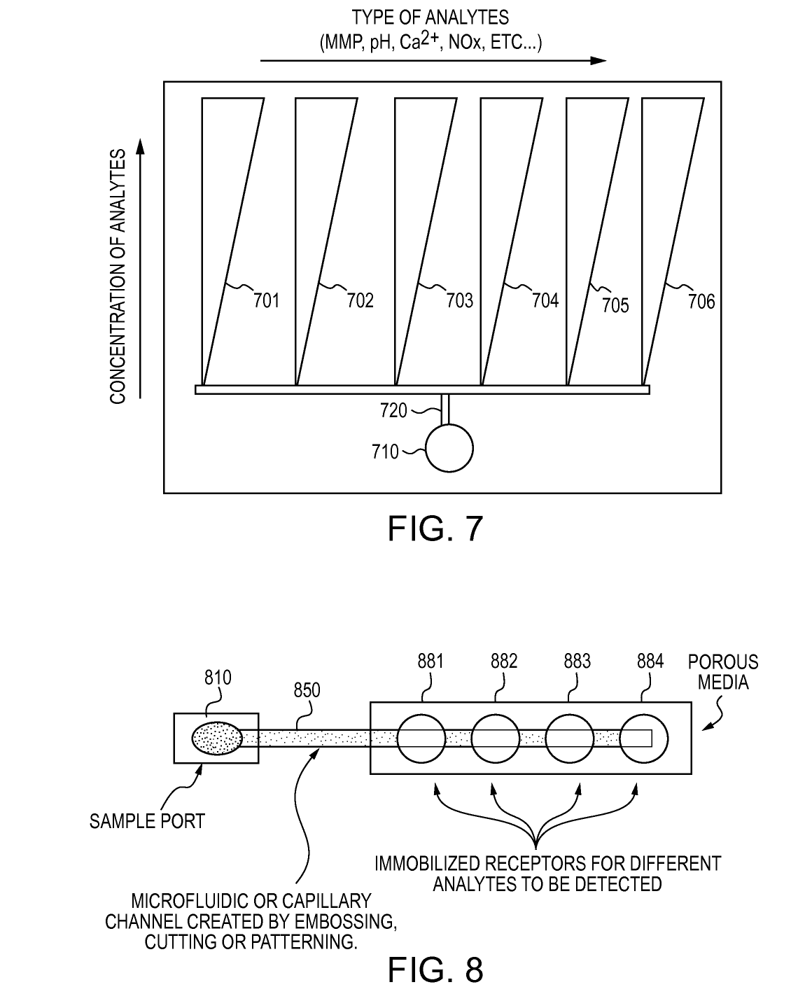

In one embodiment, the sampling layer 103 includes multiple channels with each channel feeding into a specific analyte test site. FIG. 6 is a schematic illustration of such a configuration. Each of the sampling channels 601 through 606 feeds the wound exudates to a specific analyte test site. In another embodiment of the invention as shown in FIG. 7, all of the sampling channels 701 through 706 use the same sampling port 720. As illustrated in FIG. 8, a single sampling channel 850 can also be used for multiple test sites 881 through 884 that are arranged sequentially along the sample path. The sample 810 reacts with each of the test sites as it passes through them. Preferably, the wound healing indicator device as schematically illustrated in FIG. 8 comprises a sampling layer that includes a wound location region for placement over a wound, a microfluidic sample channel, and a sample port between the wound location and the microfluidic sample channel. The device further comprises a plurality of test sites, each containing immobilized receptors for detecting one or more analytes of interest. In certain embodiments, the test sites are arranged sequentially along the microfluidic sample channel. It is also preferred that in other embodiments, multiple microfluidic sample channels are provided in a parallel configuration and each serves as a test site.

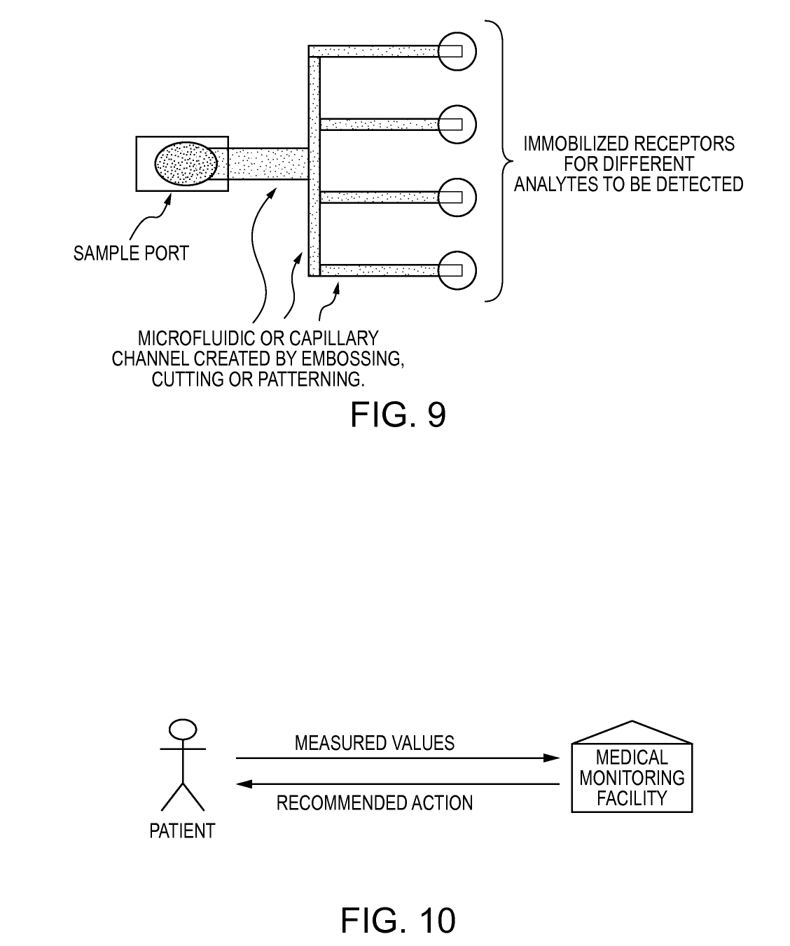

To accomplish fluid handling within the sensing device, a multitude of channels or other microfluidic components may additionally be used. Such components may include microchannels, microvalves, micromixers, and the like. The microfluidics ensemble may additionally include porosity controlled channels to separate the fluid into fractions of molecular weight, hydrodynamic radius, charge and such. Such fractions can be beneficially used to detect additional chemical or biochemical parameters with high sensitivity and low interference. The microfluidic components or capillary channels can be created by embossing, cutting or patterning techniques. Additionally, such components could be fabricated within the sensing device using roll-to-roll or moving web based manufacturing processes to increase throughput and reduce cost of such manufacturing. FIG. 9 is a schematic illustration of a design with multiparameter sensing realized through microfluidics or capillary channels.

Porosity controlled channels can be created by use of porous materials or membranes. Examples of such materials include glass frits, glass fibers, nitrocellulose membranes, polyurethane foams, polyethylene foams and the likes. Commercial sources of porous membrane and materials include Whatman (UK) and Millipore Inc. (USA).

Additional fluid extraction mechanisms that can be employed in the preferred sensing patches include iontophoresis, reverse-iontophoresis, electrokinetic and related mechanisms. Additional fluid transport mechanisms that can be beneficially employed for fluid transport in the preferred patches include electrophoresis, capillary electrophoresis and related techniques. As described herein, a number of electrical and/or mechanical stimuli can be used for extraction and handling of the fluid from a wound.

In one preferred embodiment, the visual feedback can be properly calibrated and captured by a wireless mobile device and data can be sent wirelessly to a medical practitioner's office for analysis and further action. Such devices include a cell phone, smart phone, wireless router and the likes, or a stand alone device, which takes a snapshot of the visual feedback, processes the image, compares the results with a predetermined grid, and sends the information to a medical professional if needed.

Optionally, the wound sensing device may include another component, such as an RFID device. In one preferred embodiment, the wound sensing device is coupled to a RFID device for on-demand interrogation and data transfer to a hand held reader. The RFID device can be an active or a passive device for a plurality of functions and use scenarios. In one embodiment, the detection in the described patch can utilize a light source or an electromagnetic source such as an RFID antenna. Non-limiting examples of useful wireless technologies in this context include Wi-Fi, Zigbee, BLUETOOTH®, BLE and RF based communication protocols.

Optionally, the device includes a binder component, such as a hydrogel or porous materials. A hydrogel is defined herein as a polymeric material which exhibits the ability to swell in water and retain a significant fraction, for example, more than 20%, of water within its structure but which will not dissolve in water. Synthetic and modified biopolymer hydrogels are used for numerous biomedical applications as in wound dressings, tissue regeneration and drug delivery applications among others. Examples of natural occurring hydrogels include modified collagen, modified-dextran etc. Examples of synthetic hydrogels useful in medical applications include poly (hydroxyalkyl methacrylates); poly (ethylene glycol); poly (propylene glycol); poly (acrylamide); poly (methacrylamides) and derivatives; poly (vinyl alcohol); anionic and cationic hydrogels; and poly (N-vinyl pyrrolidone) hydrogels, etc. By varying the surface/bulk charge, hydrophilicity or hydrophobicity of the monomer and the degree of crosslinking, one can vary the pore size and moisture content of hydrogels.

In one particular embodiment, the fluid (wound fluid) handling, separation and detection can be achieved by immobilizing appropriate functional components within a natural, synthetic or modified hydrogel. The inclusion or exclusion of proteins, bacteria, functional components etc. and their intake concentration can be controlled by varying the monomer type (hydrophilic/hydrophobic), monomer pendant functionality and degree of polymer crosslinking within a hydrogel. A self-contained ELISA type or related optical assay can be run within a suitably modified analyte specific hydrogel. Additional details with regard to preferred embodiments are set forth in “Biomedical Applications of Hydrogels Handbook” Raphael M. Ottenbrite, Kinam Park, Teruo Okano (Ed.) Springer Publications, 2010, pp 19-41, pp 45-63, pp 65-84, and pp 107-117.

Though illustrated as positioned on top of each other in FIG. 1, each of the components of the preferred wound sensing device, especially the functional layer and the sampling layer, can be arranged sequentially in the same layer. The sampling layer may be eliminated for applications without a need to control fluid distribution over the sampling device. The carrier layer may be eliminated when the functional layer can sustain itself. Thus, it will be appreciated that the present invention includes a wide array of alternative embodiments and configurations besides the version schematically depicted in FIG. 1.

In one preferred embodiment, the sensing device is provided in the form of a medical patch. In a further preferred embodiment, the medical patch uses multiple (two or more) sensing devices that permit the user or practitioner to assess the wound state at different times. For example, a first sensor can be activated at a first time to assess the wound state. Once the status is confirmed as satisfactory, the patch is left in place. Subsequently, a second sensor can be activated to assess the wound state at a later time and so on. The sensing device can be included in a wound care bandage as well.

A multiday use device can be provided by using a parallel array of multiple independent sensors in the patch. A particular sample port is activated by pulling an activation tab to thereby complete the fluidic circuit and provide the intended sensing analysis. As an example, an array of seven sampling ports can be incorporated in the sensing patch. Each port being designated for a different day of analysis in a given week. The activation mechanism can be through a pump action or through vacuum creation or through electrical stimulation.

It will be appreciated that wound sensing is preferably performed by a chemical sensing device which measures a plurality of analytes. Several of such analytes can indicate the general well being of the patient beyond the condition of the wound.

Patients with chronic wounds generally have one or more co-morbidities such as a history of heart or lung disease, diabetes, vascular diseases, among others. Hence it is beneficial to monitor physiological parameters in such patients while providing the treatment or advanced therapy to understand if lack of healing is due to co-morbidity and whether a particular treatment course is working.

Physiological sensors can be a part of the preferred embodiment sensing patches or can be a tandem device which is connected to the wound sensing device. The connection provides for either a time resolved or context based assessment of data from the various sensing elements.

For example, a poor activity profile or lack of sleep in a patient with a vascular disease can indicate why the particular patient's chronic wound has not shown any progress in healing since the last appointment with the wound care specialist. In this particular case, activity can be monitored by a 3-axis accelerometer using time as one of the variables. Lack of sleep can be monitored by a simple ECG measurement (continuous or intermittent) or non-activity shown in the accelerometer data over a continuous time period. Readout from the wound status indicator device after analyzing the fluid concentrations can either support or rule out any additional complications to the treatment course. A higher than normal temperature reading (in another example) can indicate infection.

A physiological sensing device when used in tandem with a wound sensing device can provide valuable historical data that will help physicians or medical specialists make accurate diagnosis. Such a tandem device can additionally empower patients or their care providers to seek early intervention to their problem.

A number of physiological parameters are used to monitor healthy lifestyle and general well being of individuals. Examples of physiological parameters, that are monitored in the medical context include electrocardiogram (ECG or EKG), blood pressure, beat, heart rate, respiration, lung volume, blood circulation, body temperature, oxygen saturation, gait, activity etc. depending on the context and prognosis. In situations of non-medical monitoring such as athletic, exercise, or weight loss related activities, these parameters provide insight into usefulness of such activities. In the case of medical monitoring, these parameters can provide life saving information such as emergency intervention, adjustment of medication or response of a patient during the course of an acute care. Acute care generally refers to outpatient, in-hospital or life threatening emergency intervention procedures. A number of such devices have been developed by several manufacturers to provide physiological monitoring of patients in acute care or hospital settings. Notable examples include the monitoring systems provided by Siemens AG, GE healthcare, Welch Allyn Inc among others. Managing chronic conditions such as diabetes, heart conditions, high blood pressure etc., on the other hand are the biggest challenge, both from care and cost considerations. Recent innovations in battery power, microprocessor and wireless technologies have led to a number of proposals for the development of simple, portable, wireless monitoring devices, see for example “Body Sensor Network” Guang-Zhong Yang (Ed.), Springer Publications, 2006, Chapter 11 and 12. In fact, a number of disclosures specifically address hardware, algorithm, power management, wireless/internet infrastructure of such physiological monitoring devices. However, there remains a need for linking such physiological sensors or devices to a medical condition so that a beneficial or actionable outcome could be achieved.

In one embodiment, the data from sensors are collected and integrated over time to provide a current status along with any future trend(s). In yet another embodiment, individual sensors in the sensing patch are used to report prevailing concentration or reporting history.

The preferred embodiment sensing patch can be used in tandem with a healing modality to indicate whether a particular therapeutic approach is working as intended. A number of stimuli based modalities are currently being used in healing of ulcers, and chronic wounds in susceptible populations such as those diabetes and vascular diseases. Such modalities typically consist of electrical or ultrasound energy impulses. The electrical modality can be based either on current, waveform, and voltage or based on a combination of these three. The ultrasonic modality may consist of frequency, time, waveform or a combination thereof. Often in case of advanced wound care therapy, the treatment involves using these stimuli based modalities along with skin graft, medication and treatment of underlying co-morbidities. Typically such treatments last for an extended period of time, for example from months to one or more years.

The preferred sensing patch can be used in tandem with medication. Active or passive drug delivery with transdermal or oral modes of delivery can be used. The sensing results can be used to measure the effectiveness of a particular dosing regimen or its effectiveness on a particular patient condition. The preferred sensing patch can be used in a home setting for self awareness and/or management of a chronic condition, and data can be easily uploaded to a primary care giver for future action. In cases where more than one combination of drugs are used, the patch can be used for providing insights on drug efficacy through measurement of one or more physical or chemical parameters using the preferred sensing patch. Medication can also be included in the sensing patch, and released when necessary to the wound area.

In one embodiment, when the sensing patch and its preferred embodiments are used in conjunction with a wireless intermediary device, the communication between the patch and the handheld is a closed loop communication. Closed loop communication particularly refers to the communication of measured parameter values to a remote facility through the internet for example and in return, receiving an advice or actionable instruction(s) to further improve the patient condition and treatment. The advice or actionable instruction can be returned or displayed to the wireless intermediary device or could follow through additional means such as using voicemail. The remote facility could provide care recommendation by using a live medical professional (physician, specialist, nurses, trained technicians etc.) or by using suitable screening programs. Several such screening programs are available from providers such as Siemens and GE Corporations. Remote facilities can also be part of regulated facilities sometimes referred to as an Integrated Diagnostic and Test Facility (IDTF), which have a government policy (such as Health and Human Services) definition and are required before such care costs can be reimbursed by government healthcare programs such as Medicare, Medicaid etc. FIG. 10 is an exemplary illustration of such a method. Information generated using the preferred sensing patch is sent to a professional site, such as a medical professional or a well established screening program. The information is then evaluated by the professional site. Actionable instruction is sent back to the patient or to a care giver through the hand held device, if needed. The patient and care giver can then follow the instruction. This process can also be carried out through the internet.

In cases where the patch utilizes an on-board processor and a power source, the battery can be a coin cell, thin-film printed or combination thereof. The power source can additionally be based on an electromagnetic energy harvesting mechanism. For example, the power could originate from the small voltage generated during the interrogation of passive RFID tags in the presence of an RFID reader.

The sensing patch system can be linked and monitored during the course of care with a medical service provider, medical insurance, public health system (for example, Medicare in USA) and the like. A financial incentive may be provided for using such patches for prevention (in some cases) or care compliance, in case of actual treatment. The reimbursement entity (or provider) can lower the cost of treatment and care by using sensing patches as a smart treatment aid.

The patch based chemical and physical sensors can be used beneficially to improve patient outcome or compliance in case of trauma or chronic conditions requiring monitoring of the previously noted indications. The patch based sensing elements, e.g. physical and/or chemical, can be used in a hospital, nursing home, long term care or home care situations by beneficially using wireless technologies to communicate information to and from the patient to a physician or care provider.

The sensing patch can be attached to a patient's wound site through the use of a pressure sensitive adhesive, an activatable adhesive, or other fastening means such as a string or hook-and-loop fasteners (also known as VELCRO).

All of the features disclosed in the specification, including the claims, abstract, and drawings, and all of the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise. Thus, unless expressly stated otherwise, each feature disclosed is one example only of a generic series of equivalent or similar features.

The foregoing detailed description of the present invention is provided for purposes of illustration, and it is not intended to be exhaustive or to limit the invention to the particular embodiments disclosed. The embodiments may provide different capabilities and benefits, depending on the configuration used to implement the key features of the invention. Accordingly, the scope of the invention is defined only by the following claims.

Many other benefits will no doubt become apparent from future application and development of this technology.

All patents, published applications, and articles noted herein are hereby incorporated by reference in their entirety.

It will be understood that any one or more feature or component of one embodiment described herein can be combined with one or more other features or components of another embodiment. Thus, the present invention includes any and all combinations of components or features of the embodiments described herein.

As described hereinabove, the present invention solves many problems associated with previous type devices, systems, and practices. However, it will be appreciated that various changes in the details, materials and arrangements of components and/or operations, which have been herein described and illustrated in order to explain the nature of the invention, may be made by those skilled in the art without departing from the principle and scope of the invention, as expressed in the appended claims.

What is claimed is:

1. A wound healing indicator device comprising:
a functional layer, wherein the functional layer determines the level of at least one specific parameter from wound exudates and provides an indication as to the status of a wound healing process.

2. The wound healing indicator device of claim 1, further comprising a carrier layer.

3. The wound healing indicator device of claim 1, further comprising a sampling layer.

4. The wound healing indicator device of claim 3, wherein the sampling layer operates through at least one of an electrophoretic mechanism, an electrokinetic mechanism, a capillary effect, a pressure driven mechanism, and combinations thereof.

5. The wound healing indicator device of claim 1, wherein the at least one specific parameter includes concentration of chemical or biochemical agents associated with at least one of bacteria, inflammation cytokines, proteases, growth factors, ECM receptors, pH, iconicity, NOₓ/O₂, temperature, integrins, and combinations thereof.

6. The wound healing indicator device of claim 1, wherein the status of the wound healing process is indicated through at least one of a color change, a digital display, and combinations thereof.

7. The wound healing indicator device of claim 6, wherein the color change is provided by use of at least one of a coulometric assay, a luminescence assay, an electrochemical assay, and combinations thereof.

8. The wound healing indicator device of claim 1, further comprising a handheld device, wherein the status of the wound healing process is received by the handheld device.

9. The wound healing indicator device of claim 8, wherein the handheld device sends information as to the status of the wound healing process to a medical monitoring facility wirelessly or through internet.

10. The wound healing indicator device of claim 1, wherein the wound healing indicator device is included in a wound care bandage.

11. A wound healing indicator patch adapted for attachment to a user, the patch comprising a wound healing indicator device, wherein the wound healing indicator device comprises:
a functional layer, wherein the functional layer determines the level of at least one specific parameter from wound exudates and provides an indication as to the status of a wound healing process.

12. The wound healing indicator patch of claim 11, further comprising at least one sensor for sensing physical characteristics associated with the user, the physical characteristics including at least one of heart rate, respiration rate, blood pressure, ECG, oxymetry, beat, activity, sleep pattern and combinations thereof.

13. The wound healing indicator patch of claim 11, further comprising a carrier layer.

14. The wound healing indicator patch of claim 13, further comprising fastening means.

15. The wound healing indicator patch of claim 14, wherein the fastening means is selected from the group consisting of a pressure sensitive adhesive, an activatable adhesive, a physical fastening means, and combinations thereof.

16. The wound healing indicator patch of claim 11, further comprising at least one of a medication and a healing modality.

17. A method of monitoring a wound healing status of a patient with a wound area, the method comprising the steps of:
applying a wound healing patch with a wound healing indication device on the wound area;
selectively activating the wound healing indication device; and
reading the results from the wound healing indication device.

18. The method of claim 17, further comprising:
sending the results to a medical monitoring facility through wireless or internet communication.

19. The method of claim 18, further comprising:
releasing medication to the wound area.

20. The method of claim 18, further comprising:
receiving advice through wireless or internet communication.

21. A method of monitoring compliance of a patient having a wound, the method comprising:
applying a wound sensing device on the wound; and
financially rewarding or penalizing the patient according to the degree of patient compliance.

22. A wound healing indicator device comprising:
a functional layer adapted for positioning on a wound;
a colored film strip disposed on the functional layer, the strip including at least a first region having a first color and a second region having a second color different than the first color.

23. The wound healing indicator of claim 22 wherein the strip includes a wound location region for placement over the wound, and the first region is located between the wound location and the second region.

24. The wound healing indicator of claim 22 further comprising a standard indicator line positioned alongside at least a portion of the colored film strip.

25. The wound healing indicator of claim 22 wherein the functional layer includes at least one functional component.

26. The wound healing indicator of claim 25 wherein the functional component reacts selectively with relevant analytes in the wound.

27. The wound healing indicator of claim 26 wherein the functional component triggers an indication component to provide indication as to wound healing.

28. The wound healing indicator of claim 25 wherein the functional component responds to at least one factor selected from the group consisting of proteases, bacterial load, inflammation cytokines, biofilm presence, moisture content, integrins, chemokines, growth factor receptors, level of Ca²⁺/Mg²⁺, NOₓ/O₂, pH, temperature, and combinations thereof.

29. A wound healing indicator device comprising:
a sampling layer including a wound location region for placement over a wound, a microfluidic sample channel, and a sample port extending between the wound location region and the microfluidic sample channel;
a means for establishing a pressure gradient in the channel upon placement of the indicator device over a wound.

30. The wound healing indicator device of claim 29 wherein the means for establishing a pressure gradient includes a tab in communication with the microfluidic sample channel, wherein upon placement of the indicator device over a wound and removal of the tab from the sample channel, a pressure gradient forms in the sample channel.

31. The wound healing indicator device of claim 29 wherein the means for establishing a pressure gradient is selected from physical rupture of a wall of the microfluidic sample channel, and applying an electric field across the microfluidic sample channel to induce transport of charged particles across the sample channel.

32. The wound healing device of claim 29 further comprising a plurality of microfluidic sample channels in the sampling layer, each sample channel configured to direct wound exudates to a different analyte test site.

33. The wound healing device of claim 32 further comprising a plurality of sampling ports, each sampling port disposed between the wound location region and a corresponding microfluidic sample channel.

34. The wound healing device of claim 32 further comprising a single sample port providing communication between the wound location region and the plurality of microfluidic sample channels.

35. The wound healing device of claim 29 further comprising a plurality of test sites each in communication with the microfluidic sample channel and the plurality of test sites arranged sequentially along the sample channel.

36. The wound healing device of claim 35 wherein the plurality of test sites include immobilized receptors for detecting one or more analytes.

37. The wound healing device of claim 29 further comprising a plurality of test sites and a plurality of microfluidic sample channels, wherein each sample channel provides communication between the wound location region and a corresponding test site.

38. The wound healing device of claim 37 wherein the plurality of test sites include immobilized receptors for detecting one or more analytes.
